(19) United States
(12) Patent Application Publication
(10) Pub. No.: US 2024/0069000 A1
(43) Pub. Date: Feb. 29, 2024

(54) NON-INVASIVE METHOD OF DETERMINING PROPERTIES OF A CHICKEN EGG AND/OR PROPERTIES OF A CHICKEN EMBRYO INSIDE THE EGG USING NEAR IR SPECTROSCOPY, RESPECTIVE SYSTEM AND USES THEREOF

(71) Applicant: AGRI ADVANCED TECHNOLOGIES GMBH, Visbek (DE)

(72) Inventor: Jörg HURLIN, Dötilngen (DE)

(73) Assignee: AGRI ADVANCED TECHNOLOGIES GMBH, Visbek (DE)

(21) Appl. No.: 18/258,287

(22) PCT Filed: Dec. 17, 2021
(86) PCT No.: PCT/EP2021/086509

§ 371 Date: Jun. 19, 2023

(30) Foreign Application Priority Data
Dec. 17, 2020 (EP) 20215032.2

(51) Int. Cl. G01N 33/08; A61B 5/00; G01N 21/95

(52) U.S. Cl. CPC G01N 33/085; A61B 5/0075; G01N 21/95; A61B 2503/40; G01N 2201/126

(57) ABSTRACT

Described is a non-invasive method of determining one or more properties of an avian egg, in particular a chicken egg, and/or one or more properties of an avian embryo, in particular a chicken embryo, inside the egg, using near IR spectroscopy. More specifically, it is described herein a non-invasive method of determining the sex of an avian embryo, in particular a chicken embryo, inside the egg. Furthermore described herein is a system for non-invasively determining one or more properties of an avian egg, in particular a chicken egg, and/or one or more properties of an avian embryo, in particular a chicken embryo, inside the egg, as well as the use of a spectrometer selected from a Multi Channel Spectrometer, a Compact Grating Spectrometer and a Monolithic Miniature Spectrometer, in a system described herein.

The present invention relates to a non-invasive method of determining one or more properties of an avian egg, in particular a chicken egg, and/or one or more properties of an avian embryo, in particular a chicken embryo, inside the egg, using near IR spectroscopy. More specifically, the present invention pertains to a non-invasive method of determining the sex of an avian embryo, in particular a chicken embryo, inside the egg. The present invention further relates to a system for non-invasively determining one or more properties of an avian egg, in particular a chicken egg, and/or one or more properties of an avian embryo, in particular a chicken embryo, inside the egg, as well as to the use of a spectrometer selected from a Multi Channel Spectrometer, a Compact Grating Spectrometer and a Monolithic Miniature Spectrometer, in a system and/or in a method of the present invention.

Production of avian eggs, in particular of chicken eggs (synonymously used herein for the term “hen's eggs”), for human food consumption or for chicken breeding is today often performed on an industrial basis. Due to the large amount of eggs produced, quick and reliable methods for controlling quality and properties of the eggs and of the embryos inside the eggs are required. Another industrial area where avian eggs, in particular chicken eggs, of controlled quality are needed, is the production of vaccines, in particular of influenza vaccines. The so-called “egg-based vaccine manufacturing process” is currently used to make inactivated vaccine as well as live attenuated vaccine.

In egg production, chicken breeding and in the egg-based vaccine manufacturing process there is therefore a vital interest in obtaining quick and reliable information on properties of an egg, e.g. an egg's fertilisation state (fertilised or unfertilised) or an egg's (internal) germ load. Primarily in chicken egg production and chicken breeding, there is also a more specific interest in the properties of the chicken embryo inside the egg, e.g. the chicken embryo's state of development, the vitality of the chicken embryo and the sex of the chicken embryo. Once the properties of a chicken egg or of a chicken embryo inside the egg have been determined, the eggs comprising chicken embryos with desired properties can be identified and used further.

For example, breeds of hens bred for the production of eggs are usually less suitable for the production of meat for reasons of economy and product quality. Male chicks of these laying breeds are therefore in most cases not reared and are currently often sacrificed after hatching. Since this current practice is not only unsatisfactory from an economic standpoint but also highly undesirable from an ethical perspective, efforts have been made for some time to avoid the culling of male chicks after hatching.

Several publications already deal with methods of hatching avian eggs, including determining one or more properties of an avian egg and/or one or more properties of an avian embryo inside the egg, the following being among them:

Document U.S. Pat. No. 5,575,237 discloses a method of hatching avian eggs.

Document WO 2010/150265 A2 pertains to hyperspectral identification of egg fertility and gender.

Document WO 2014/033544 A2 deals with spectrophotometric analysis of embryonic chick feather colour.

Document WO 2019/174661 A1 describes a device for examining hatching eggs.

D. Göhler et al. in Poultry Science (2017) 96 (1): 1-4 report of in-ovo sexing of 14-day-old chicken embryos by pattern analysis in hyperspectral images (VIS/NIR spectra)—a non-destructive method for layer lines with gender-specific down feather colour.

In the light of the existing prior art there is still a need fora convenient, quick and reliable method of determining one or more properties of an avian egg, in particular of a chicken egg and/or one or more properties of an avian embryo, in particular a chicken embryo, inside the egg.

Correspondingly, it was a primary object of the present invention to provide a convenient, quick and reliable method of determining one or more properties of an avian egg, in particular a chicken egg, and/or one or more properties of an avian embryo, in particular a chicken embryo, inside the egg. A more specific object of the present invention pertained to a non-invasive method of determining the sex of an avian embryo, in particular a chicken embryo, inside the egg.

It was another object of the present invention to provide a simple and inexpensive system for non-invasively determining one or more properties of an avian egg, in particular of a chicken egg, and/or one or more properties of an avian embryo, in particular of a chicken embryo, inside the egg.

A further object of the invention related to expanding the field of applications of certain types of spectrometers.

It has now been found that the primary object and other objects of the present invention can be accomplished by a non-invasive method of determining one or more properties of an avian egg, preferably of a chicken egg, and/or one or more properties of an avian embryo, preferably of a chicken embryo, inside the egg, comprising at least the following steps:
M1) obtaining an avian egg, preferably an egg from a chicken (hen),
M2) candling the egg obtained in step M1) with light having a spectrum extending at least over the range of wavelengths from ≥700 nm to ≤900 nm from a light source,
M3) capturing light transmitted through the egg, wherein the captured transmitted light is a portion of the light used for candling the egg in step M2),
M4) acquiring a transmission spectrum of the transmitted light captured in step M3) based on one or more distinctive wavelength ranges, wherein the one or more distinctive wavelength ranges are in each case predefined wavelength subranges of the range of wavelengths from ≥700 nm to ≤900 nm as defined in step M2)
and
M6) determining the one or more properties of the avian egg, preferably of the chicken egg, and/or the one or more properties of the avian embryo, preferably the chicken embryo, inside the egg, based on the transmission spectrum acquired in step M4), preferably solely based on the one or more distinctive wavelength ranges of the transmission spectrum.

The invention as well as preferred variants and preferred combinations of parameters, properties and elements thereof are defined in the appended claims. Preferred aspects, details, modifications and advantages of the present invention are also defined and explained in the following description and in the examples shown below.

It has been found in own experiments that the non-invasive method of the present invention is excellently suited for detecting or determining an unhatched avian (preferably chicken) egg's fertilization state (fertilized or unfertilized), an unhatched avian (preferably chicken) egg's (internal) germ load, an unhatched avian (preferably chicken) embryo's vitality inside the egg, an unhatched avian (preferably chicken) embryo's state of development (inside the egg) and an unhatched avian (preferably chicken) embryo's sex.

In step M1) of the non-invasive method according to the invention, an avian egg is obtained. Preferred is an egg from a chicken (i.e. a hen's egg). In preferred variants of the method of the present invention, the egg is obtained from a breed of chicken that produces brown or brownish feathers for one sex and white or yellowish feathers for the opposite sex, as explained in more detail below.

In step M2) of the non-invasive method according to the invention, the egg is candled with light having a spectrum extending at least over the range of wavelengths from ≥700 nm to ≤900 nm from a light source. Preferably, the egg is candled from below. It is preferred that the light from the light source is transmitted through the egg along its major axis or along its minor axis.

In step M3) of the non-invasive method according to the invention, the light which has been transmitted through the egg is captured, preferably by light capturing means as described below. The captured transmitted light is a portion of the light used for candling the egg in step M2), where preferably the remaining light portion (which is not captured), or at least a major part of the remaining light portion, has been absorbed by the egg or the egg's content. In this case, the egg might be candled such that the light from the light source used for candling directly enters the eggs. However, in other cases, the light source used for candling might be positioned at a non-zero distance to the egg, such that the light from the light source used for candling does not directly enter the egg. More generally, after entering the egg, some of the light might leave the egg again without being captured, particularly if the light is captured not directly on the egg's surface and/or the light used for candling the egg is not a beam of light focused and/or directed at where the light is captured. Hence, it can also be preferred that the remaining light portion referred to above, i.e. the light which is not captured, comprises light which has been absorbed by the egg or the egg's content and light which has not entered the egg or has entered the egg but has left the egg without being captured.

In step M4) of the non-invasive method according to the invention, a transmission spectrum is acquired of the transmitted light captured in step M3). The transmission spectrum is preferably acquired by a suitable spectrometer to which the transmitted light is preferably guided by suitable light guiding means, as described in more detail below. Acquiring the transmission spectrum of the transmitted light captured in step M3) based on the one or more distinctive wavelength ranges can mean, in an example, that the acquired spectrum only contains the distinctive wavelength ranges. More generally, the transmission spectrum can be acquired with an accuracy that is higher in the distinctive wavelength ranges than elsewhere in the acquired transmission spectrum, wherein the accuracy can refer, for instance, to an absence of losses as determined with respect to the transmitted light, or a faithfulness of the acquired spectrum with respect to the actual, or true, spectrum of the transmitted light. In fact, acquiring the transmission spectrum of the transmitted light captured in step M3) based on the one or more distinctive wavelength ranges can also mean that the acquired transmission spectrum contains at least the distinctive wavelength ranges, preferably with a sufficient accuracy and/or faithfulness. In any case, how the transmission spectrum is acquired, and hence the acquired transmission spectrum, will generally depend on specifications of the technical means used for acquiring the transmission spectrum, i.e., preferably the spectrometer, but also the light guiding means, for instance. These technical means, i.e. particularly the spectrometer and/or the light guiding means, for instance, might therefore be adapted to allow for an acquisition of the transmission spectrum based on the distinctive wavelength ranges.

In step M6) of the non-invasive method according to the invention, one or more properties of the avian egg, preferably of the chicken egg, and/or of the one or more properties of the avian embryo, preferably the chicken embryo, inside the egg, are determined. Preferably, a determination unit (as described in more detail below) is used for determining said properties in step M6) (including in preferred variants of step M6) as described herein). The determination unit preferably comprises a data processing unit which is adapted for this purpose. Preferably, the data processing unit also comprises a software suitable for the purposes according to the present invention.

Preferred is a method according to the present invention (or a method according to the present invention as described herein as being preferred),
wherein the one or one of the more properties of the avian egg, preferably of the chicken egg, is selected from the group consisting of the egg's fertilization state and the (internal) germ load of the egg;
   and/or
wherein the one or one of the more properties of the avian embryo, preferably of the chicken embryo, inside the egg is selected from the group consisting of the avian embryo's, preferably of the chicken embryo's, state of development, the vitality of the avian embryo, preferably of the chicken embryo, and the sex of the avian embryo, preferably of the chicken embryo;
   and/or
comprising as additional step M5) the following step:
M5) comparing the transmission spectrum of the transmitted light acquired in step M4), or an absorption spectrum based on said transmission spectrum, in the one or more distinctive wavelength ranges with corresponding transmission spectra or with corresponding absorption spectra from a predefined database in the respective same one or more distinctive wavelength ranges (preferably only in the one or more defined distinctive wavelength ranges), where the corresponding transmission spectra or the corresponding absorption spectra, particularly in the distinctive wavelength ranges, define known values of the one or more properties of the avian egg, preferably of the chicken egg and/or of the one or more properties of the avian embryo, preferably of the chicken embryo, inside the egg;
   and/or
comprising as step M6) the following step:
M6) determining the one or more properties of the avian egg, preferably of the chicken egg, and/or the one or more properties of the avian embryo, preferably the chicken embryo, inside the egg, based on the transmission spectrum acquired in step M4) and/or based on the result of comparing the transmission spectrum of the transmitted light acquired in step M4), or the absorption spectrum based on said transmission spectrum, in the one or more defined distinctive wavelength ranges (preferably solely in the one or more distinctive wavelength ranges) with the corresponding transmission spectra or with the corresponding absorption spectra from a predefined database in the respective same one or more distinctive wavelength ranges, as defined in step M5).

In step M5) of the non-invasive method according to the invention, a transmission spectrum of the transmitted light acquired in step M4), or an absorption spectrum based on said transmission spectrum, is compared in the one or more distinctive wavelength ranges with corresponding transmission spectra or with corresponding absorption spectra from a predefined database in the respective same one or more distinctive wavelength ranges, where the corresponding transmission spectra or the corresponding absorption spectra define known values of the one or more properties to be determined (as described above or below). Preferably, the determination unit (as described in more detail below) is used for comparing the spectra (as described above and below) in step M5) (including in preferred variants of step M5) as described herein). The determination unit preferably comprises a data processing unit which is adapted for this purpose. Preferably, the data processing unit also comprises a software suitable for the purposes according to the present invention.

The comparison according to step M5) allows for an efficient determination of the one or more properties of interest, since, instead of the whole spectra, only parts of the spectra are compared, such that less data need to be processed. Preferably, the distinctive wavelength ranges are predetermined based on the type of property that is to be determined, i.e. also the type of eggs and/or avian species, and the acquired transmission spectra from the predefined database, which might be considered as forming the training data. An analysis of the training data, which comprises spectra acquired for eggs where the property to be determined is known (generally by use of other means than spectral analysis, such as conventional sex determination of chicks after hatching in case the property to be determined is the sex of the embryo inside the egg), this knowledge being part of the training data, can reveal that certain wavelength ranges in the spectra are particularly indicative of the property to be determined, wherein these wavelength ranges may then be chosen as the distinctive wavelength ranges. This analysis can be executed by a human being, or by an artificial intelligence. For instance, a machine learning architecture may be trained on the predefined database of spectra to receive a spectrum acquired as defined in steps M1) to M4) (as described above or below), specifically also a spectrum not in the database, as an input, and to provide the one or more properties to be determined for the respective egg and/or embryo inside the egg as an output. However, the comparing as referred to in step M5) may be realized in many other ways as well. Generally, the comparing might be understood, for instance, as determining a degree of similarity between a spectrum acquired as defined in steps M1) to M4) (as described above or below), specifically also a spectrum not in the database, with the corresponding spectra from the database in the respective same one or more distinctive wavelength ranges. If the degree of similarity exceeds a given threshold, the values of the one or more properties defined by the respective spectrum from the database might be assumed to hold also for the acquired spectrum. For measuring the degree of similarity, basically any measure of similarity between two (discrete) functions can be used.

The non-invasive method according to the present invention is particularly preferred for determining one or more properties of a chicken egg, and/or one or more properties of a chicken embryo inside the egg.

In step M5) as defined here above, it is preferred according to the method of the present invention to compare an absorption spectrum based on the transmission spectrum of the transmitted light acquired in step M4) in the one or more distinctive wavelength ranges with corresponding absorption spectra from a predefined database in the respective same one or more distinctive wavelength ranges (preferably only in the one or more defined distinctive wavelength ranges), where the corresponding absorption spectra define known values of the one or more properties of the avian egg, preferably of the chicken egg and/or of the one or more properties of the avian embryo, preferably of the chicken embryo, inside the egg.

In step M6) as defined here above, it is preferred according to the method of the present invention to determine the one or more properties of the avian egg, preferably of the chicken egg, and/or the one or more properties of the avian embryo, preferably the chicken embryo, inside the egg, based on the result of comparing the absorption spectrum based on the transmission spectrum of the transmitted light acquired in step M4) in the one or more defined distinctive wavelength ranges (preferably solely in the one or more distinctive wavelength ranges) with the corresponding absorption spectra from a predefined database in the respective same one or more distinctive wavelength ranges as defined in step M5).

According to the method of the present invention, an “absorption spectrum” as described herein is preferably determined based on the transmission spectrum acquired in step M4) and a calibration spectrum, wherein the calibration spectrum is a measured spectrum of the light used for candling the egg in step M2), wherein preferably the transmission spectrum based on which the absorption spectrum is determined is corrected based on a dark current spectrum, wherein the dark current spectrum corresponds to a spectrum which is acquired under equal circumstances as the transmission spectrum of step M4), with the exception that no light is transmitted through the egg. In an example, an absorption spectrum I_{ab} might be computed as I_{ab}=I₀−(Iₜᵣ−I_{dark}), with I₀ referring to the calibration spectrum, Iₜᵣ referring to the acquired transmission spectrum and I_{dark} referring to the dark current spectrum. However, the absorption spectrum might also be defined, or computed, differently. In cases where the transmission spectra are acquired without any ambient light present, measuring the dark current spectrum and using it to compute the absorption spectra might not be necessary. The term “absorption spectrum” as used herein might, in fact, also refer to an absorbance, i.e. an absorbance spectrum. In an example, an absorbance spectrum, can be computed from a transmission spectrum as I_{ab}=log₁₀[I₀(Iₜᵣ−I_{dark})], with I₀ referring to the calibration spectrum, Iₜᵣ referring to the acquired transmission spectrum and I_{dark} referring to the dark current spectrum.

It has been found in own experiments that the non-invasive method of the present invention, in particular in its specific or more specific variants as defined herein, is excellently suited for determining the sex of an avian embryo, in particular of a chicken embryo, inside the egg, more in particular when eggs are used in the present method from an avian species that produces feathers of one colour for one sex and feathers of another colour for the opposite sex. A preferred example of such an avian species is a breed of chicken that produces feathers of one colour for one sex and feathers of another colour for the opposite sex, in particular a breed of chicken that produces brown or brownish feathers for one sex and white or yellowish feathers for the opposite sex. Such breeds of chicken are known in the field, e.g. the brown layer breed of chicken known as “Hyline Brown”, “Lohmann Brown” (Lohmann Tierzucht GmbH, Germany) or “ISA Brown”. The “ISA Brown” is a crossbreed of chicken, with sex-linked coloration. “ISA” stands for “Institut de Selection Animale”, the company which developed the crossbreed in 1978.

In a specific variant, a method according to the present invention is therefore preferred (or a method according to the present invention as described herein as being preferred), wherein preferably the method is a non-invasive method of determining the sex of a chicken embryo inside the egg,
preferably comprising
   as step M1) the following step:
   M1) obtaining an egg from a breed of chicken that produces feather colour differentiation of the chicken, based on the sex of the chicken,
and/or
   as additional step M5) the following step:
   M5) comparing the transmission spectrum of the transmitted light acquired in step M4), or an absorption spectrum based on said transmission spectrum, in the one or more distinctive wavelength ranges with corresponding transmission spectra or corresponding absorption spectra from a predefined database in the respective same one or more distinctive wavelength ranges, preferably only in the one or more defined distinctive wavelength ranges, where the corresponding transmission spectra or the corresponding absorption spectra define known sexes of chicken embryos inside their eggs;
and/or
   as step M6) the following step:
   M6) determining the sex of the chicken embryo inside the egg based on the transmission spectrum acquired in step M4) and/or based on the result of comparing the transmission spectrum of the transmitted light acquired in step M4), or an absorption spectrum based on said transmission spectrum, in the one or more defined distinctive wavelength ranges, preferably only in the one or more defined distinctive wavelength ranges, with corresponding transmission spectra or with corresponding absorption spectra from a predefined database in the respective same one or more distinctive wavelength ranges, as defined in step M5).

In step M5) as defined here above, it is preferred according to the method of the present invention to compare the absorption spectrum based on the transmission spectrum of the transmitted light acquired in step M4) in the one or more distinctive wavelength ranges with corresponding absorption spectra from a predefined database in the respective same one or more distinctive wavelength ranges, preferably only in the one or more defined distinctive wavelength ranges, where the corresponding absorption spectra define known sexes of chicken embryos inside their eggs.

In step M6) as defined here above, it is preferred according to the method of the present invention to determine the sex of the chicken embryo inside the egg based on the result of comparing the absorption spectrum based on the transmission spectrum of the transmitted light acquired in step M4) in the one or more defined distinctive wavelength ranges, preferably only in the one or more defined distinctive wavelength ranges, with the corresponding absorption spectra from a predefined database in the respective same one or more distinctive wavelength ranges, as defined in step M5).

In a more specific variant, a non-invasive method according to the present invention is preferred (or a method according to the present invention as described herein as being preferred), wherein the method is a non-invasive method of determining the sex of a chicken embryo inside the egg, comprising the following steps:
M1) obtaining an egg from a breed of chicken that produces feather colour differentiation of the chicken, based on the sex of the chicken,
M2) candling the egg obtained in step M1) with light having a spectrum extending at least over the range of wavelengths from ≥700 nm to ≤900 nm,
M3) capturing light transmitted through the egg, wherein the captured transmitted light is a portion of the light used for candling the egg in step M2),
M4) acquiring a transmission spectrum of the transmitted light captured in step M3) based on one or more distinctive wavelength ranges, wherein the one or more distinctive wavelength ranges are in each case predefined wavelength subranges of the range of wavelengths from ≥700 nm to ≤900 nm as defined in step M2),
M5) comparing an absorption spectrum based on the transmission spectrum of the transmitted light acquired in step M4) in the one or more distinctive wavelength ranges with corresponding absorption spectra from a predefined database in the respective same one or more distinctive wavelength ranges, preferably only in the one or more defined distinctive wavelength ranges, where the corresponding absorption spectra define known sexes of chicken embryos inside their eggs (i.e. the corresponding absorption spectra serve as reference spectra)
and
M6) determining the sex of the chicken embryo inside the egg based on the result of comparing the absorption spectrum based on the transmission spectrum of the transmitted light acquired in step M4) in the one or more defined distinctive wavelength ranges, preferably only in the one or more defined distinctive wavelength ranges, with the corresponding absorption spectra from a predefined database in the respective same one or more distinctive wavelength ranges, as defined in step M5)

Also preferred is a method according to the present invention (or a method according to the present invention as described herein as being preferred), wherein the method is a non-invasive method of determining the sex of a chicken embryo inside the egg, wherein the egg obtained in step M1)
is obtained from a breed of chicken that produces brown or brownish feathers, preferably brown or brownish down feathers, for one sex and white or yellowish feathers, preferably white or yellowish down feathers, for the opposite sex;
and/or
is obtained from a breed of chicken which is a brown layer breed of chicken, preferably selected from the group consisting of a Hyline Brown breed of chicken, a Lohmann Brown breed of chicken and an ISA Brown breed of chicken;
and/or
is a hatching egg.

It has turned out that particularly good results are received with the method according to the present invention in terms of accuracy of determining the sex of an avian embryo, in particular of a chicken embryo, inside the egg, when the feather colour of the avian embryo, in particular of the chicken embryo, has developed at the time the method is applied to an extent that allows its detection in the method with sufficient precision. In many cases, the feather colour of an avian embryo, in particular of a chicken embryo, more in particular of a chicken embryo of a chicken breed which is preferably used in the method of the present invention (as described above), has developed to a sufficient extent when the avian egg (in particular the chicken egg) has been incubated for a period in the range from ≥9 to ≤15 days, preferably in the range from ≥12 to ≤14 days, more preferably in the range from ≥13 to ≤14 days, after laying. More generally, the period for which the avian egg has been incubated before applying the method of the present invention (as described above or below) might be chosen depending on the time at which the avian embryos in the eggs develop features indicative of a property to be determined and influencing the embryo's and/or the egg's optical properties, particularly its transmission characteristics, in the one or more distinctive wavelength ranges. The down feathers of avian embryos, particularly chicken embryos, can be an example of such a feature, since their colour can depend on the sex of the embryo.

It has further been found in own experiments that (preferably in variants of the present invention relating to determining the sex of an avian embryo, in particular of a chicken embryo, inside the egg, where eggs are used from an avian species, in particular from a breed of chicken, that produces feathers of one colour for one sex and feathers of another colour for the opposite sex) the most significant spectral information is received from the wavelength range from ≥700 nm to ≤900 nm, or the preferred sub-ranges, particularly the predefined distinctive wavelength (sub-)ranges, of this range as described herein. These findings apply in particular when the eggs used in the method of the present invention are obtained from a breed of chicken that produces brown or brownish down feathers for one sex and white or yellowish down feathers for the opposite sex.

Consequently, a method according to the present invention is preferred (or a method according to the present invention as described herein as being preferred), wherein
the egg (preferably the chicken egg) obtained in step M1) has been incubated for a period in the range from ≥9 to ≤15 days, preferably in the range from ≥12 to ≤14 days, more preferably in the range from ≥13 to ≤14 days after laying;
and/or
the light used for candling the egg in step M2) is light having a spectrum extending at least over the range of wavelengths from ≥720 nm to ≤870 nm, preferably from >750 nm or ≥750 nm to ≤870 nm.

As regards capturing of the light transmitted through the egg in step M3) of the method of the present invention, own experiments have revealed that the diameter of the measuring spot on the egg's surface should be chosen large enough so that occasionally occurring inaccurate positioning of the eggs in a carrier or carrier-rack compartments (as explained below) may not negatively affect the measurement results to an extent that it would be relevant in practice. The measuring spot may be defined as a region on the egg's surface in which light which has been transmitted through the egg is captured.

Also preferred (for all variants of the method of the present invention as described herein) is therefore a method according to the present invention (or a method according to the present invention as described herein as being preferred), wherein the light transmitted through the egg is captured in step M3) within a defined measuring spot on the egg's surface, wherein the measuring spot
has a diameter in the range from ≥0.5 to ≤2.5 cm, preferably from ≥1 to ≤2.3 cm;
and/or
extends over an area in the range from ≥0.2 to ≤5 cm², preferably from ≥0.8 to ≤4 cm² on the egg's surface.

In the course of own experiments it turned out to be favourable, in particular when larger quantities of eggs are to be processed, if the eggs used in the method of the present invention are placed in separate compartments of a carrier, preferably a carrier rack, to reduce the amount of scattered light that may otherwise potentially interfere with the measurement. Preferably, partition walls, i.e. the partitioning walls of the compartments, are coloured in black to further reduce the amount of scattered light. The carrier or carrier rack is preferably configured to allow candling of the egg with a light source, preferably from below, in step M2) and capturing light transmitted through the egg, preferably from the side opposite to the side where the light source is placed (preferably from above), in step M3) of the non-invasive process according to the present invention.

Preferred is (for all variants of the method of the present invention as described herein) a method according to the present invention (or a method according to the present invention as described herein as being preferred), wherein step M1) further comprises
providing a carrier, preferably a carrier rack, having a plurality of compartments, wherein each compartment is configured for receiving an avian egg, preferably a chicken egg, and the compartments are separated from each other by partition walls for reducing the amount of scattered light,
wherein preferably the carrier, preferably the carrier rack, is configured for allowing candling with a light source an avian egg, preferably a chicken egg, placed in a compartment; and for allowing capturing light transmitted through the egg (preferably including allowing light-tight coupling of the light source to the egg);
and
placing the avian egg, preferably the chicken egg, in a compartment of the carrier, preferably of the carrier rack.

In a more specific variant of the method according to the present invention, a carrier, preferably a carrier rack, as disclosed and described in more detail in published document WO 2019/174661 A1 (as “Träger (3)”) may be used.

A suitable light source for candling in step M2) the egg obtained in step M1) of the non-invasive method according to the present invention comprises in its emitted spectrum of electromagnetic radiation light having a spectrum extending at least over the range of wavelengths from ≥700 nm to ≤900 nm, preferably a continuous spectrum extending at least over the range of wavelengths from ≥700 nm to ≤900 nm. Suitable light sources in this sense comprise incandescent light bulbs and halogen lamps, in particular tungsten halogen lamps. Usually, a light source with a power of 35 W and/or a luminous intensity of 1100 cd is suited for the purpose of the method of the present invention. In particular fora variant of the method of the present invention where the sex of an avian embryo, preferably a chicken embryo, in the egg shall be determined, a light source with a higher power and/or a higher luminous intensity may be beneficial. More powerful lamps with a higher luminous intensity, in particular of ≥35 W, preferably of ≥40 W, more preferably of ≥50 W, and preferably ≤75 W, allow shorter integration times and consequently a higher process speed.

Preferred is therefore a method according to the present invention (or a method according to the present invention as described herein as being preferred), wherein the light source for candling the egg in step M2) is a halogen lamp, preferably a tungsten halogen lamp, wherein the halogen lamp has
a power of ≥35 W, preferably of ≥40 W, more preferably of ≥50 W, and preferably ≤75 W;
and/or
a luminous intensity of ≥1000 cd, preferably of ≥1100 cd, more preferably of ≥1200 cd and even more preferably of ≥1300 cd.

It has been found in own experiments that particularly good results in terms of determining the sex of an avian embryo inside the egg, preferably of a chicken embryo inside the egg, are achieved in preferred variants of the present invention where eggs are used from avian species, preferably of breeds of chicken, that produce feathers of a certain colour for one sex and feathers of another (different) colour for the opposite sex and where the transmission spectrum of the transmitted light captured in step M3) and acquired in step M4) is based on one or more distinctive wavelength ranges, wherein the one or more distinctive wavelength ranges of step M4) comprise the wavelength ranges from ≥720 nm to ≤760 nm, from ≥730 nm to ≤830 nm, from ≥750 nm to ≤870 nm and/or from ≥800 nm to ≤870 nm, as specified in more detail below.

Moreover, particularly good results in said preferred variants of the method of the present invention in terms of determining the sex of an avian embryo inside the egg, preferably of a chicken embryo inside the egg, are achieved in more preferred variants of the present invention comprising a step M5) and said step M5) comprises comparing the transmission spectrum of the transmitted light acquired in step M4) in one or more distinctive wavelength ranges, wherein the one or more distinctive wavelength ranges are selected from the wavelength ranges from ≥720 nm to ≤760 nm, from ≥730 nm to ≤830 nm, from ≥750 nm to ≤870 nm and from ≥800 nm to ≤870 nm, with the corresponding transmission spectra defining known sexes of chicken embryos inside their eggs, in the respective same one or more distinctive wavelength ranges. These findings apply in particular when the eggs used in the method of the present invention are of a breed of chicken that produces brown or brownish down feathers for one sex and white or yellowish down feathers for the opposite sex.

Preferred is therefore a method according to the present invention (or a method according to the present invention as described herein as being preferred), wherein preferably the method is a non-invasive method of determining the sex of a chicken embryo inside the egg,
wherein
   the one or more distinctive wavelength ranges of step M4) are selected from the group consisting of
      the wavelength range from ≥720 nm to ≤760 nm
      the wavelength range from ≥730 nm to ≤830 nm,
      the wavelength range from ≥750 nm to ≤870 nm, preferably from >750 nm or 750 nm to ≤830 nm
   and
      the wavelength range from ≥800 nm to ≤870 nm,
   wherein preferably the one or more distinctive wavelength ranges of step M4) are selected from the group consisting of
      the wavelength range from ≥750 nm to ≤870 nm, preferably from >750 nm or 750 nm to ≤830 nm
   and
      the wavelength range from ≥800 nm to ≤870 nm;
and/or
   the method comprises as step M5) the following step:
   M5) comparing the transmission spectrum of the transmitted light acquired in step M4) or an absorption spectrum based on said transmission spectrum in the one or more distinctive wavelength ranges, preferably only in the one or more distinctive wavelength ranges, wherein the one or more distinctive wavelength ranges are selected from the group of wavelength ranges consisting of:
      the wavelength range from ≥720 nm to ≤760 nm
      the wavelength range from ≥730 nm to ≤830 nm,
      the wavelength range from ≥750 nm to ≤870 nm, preferably from >750 nm or 750 nm to ≤830 nm
      and
      the wavelength range from ≥800 nm to ≤870 nm,
         wherein preferably the one or more distinctive wavelength ranges of step M4) are selected from the group consisting of
         the wavelength range from ≥750 nm to ≤870 nm, preferably from >750 nm or ≥750 nm to ≤830 nm
         and
         the wavelength range from ≥800 nm to ≤870 nm,
      with corresponding transmission spectra or corresponding absorption spectra from a predefined database in the respective same one or more distinctive wavelength ranges, where the corresponding transmission spectra or the corresponding absorption spectra define known sexes of chicken embryos inside their eggs;
and/or
   the method comprises as step M6) the following step:
   M6) determining the sex of the avian embryo, preferably of the chicken embryo, inside the egg, based on the result of comparing, preferably of comparing only,
      the transmission spectrum of the transmitted light acquired in step M4) in the one or more defined distinctive wavelength ranges or an absorption spectrum based on said transmission spectrum, in the one or more defined distinctive wavelength ranges, as defined in step M5),
      with
      corresponding transmission spectra or with corresponding absorption spectra defining known sexes of chicken embryos inside their eggs in the respective same one or more distinctive wavelength ranges, as defined in step M5).

In step M5) as defined here above, it is preferred according to the method of the present invention to compare the absorption spectrum based on the transmission spectrum of the transmitted light acquired in step M4) in the one or more distinctive wavelength ranges (as defined above), preferably only in the one or more distinctive wavelength ranges, with corresponding absorption spectra from a predefined database in the respective same one or more distinctive wavelength ranges, where the corresponding absorption spectra define known sexes of chicken embryos inside their eggs.

In step M6) as defined here above, it is preferred according to the method of the present invention to determine the sex of the chicken embryo inside the egg, based on comparing, preferably of comparing only, the absorption spectrum based on the transmission spectrum of the transmitted light acquired in step M4) in the one or more defined distinctive wavelength ranges as defined in step M5), with corresponding absorption spectra defining known sexes of chicken embryos inside their eggs in the respective same one or more distinctive wavelength ranges, as defined in step M5)

For example, in the preferred variant of the method of the present invention as defined above, for determining the sex of the avian embryo, preferably of the chicken embryo, inside the egg, in step M6), the transmission spectrum of the transmitted light acquired in step M4) in the wavelength range from ≥800 nm to ≤870 nm is compared with corresponding transmission spectra defining known sexes of chicken embryos inside their eggs in the same wavelength range from ≥800 nm to ≤870 nm.

For further improving the results of the method according to the present invention, it is preferred to take a calibration spectrum and/or a dark current spectrum.

Preferred is a method according to the present invention (or a method according to the present invention as described herein as being preferred, preferably a method of determining the sex of a chicken embryo inside the egg) wherein the method comprises step M5) and wherein in step M6) determining the sex of the avian embryo, preferably the sex of the chicken embryo, is based on an absorption spectrum, wherein the absorption spectrum is determined based on the transmission spectrum acquired in step M4) and a calibration spectrum, wherein the calibration spectrum is a measured spectrum of the light used for candling the egg in step M2),
wherein preferably the transmission spectrum based on which the absorption spectrum is determined is corrected based on a dark current spectrum, wherein the dark current spectrum corresponds to a spectrum which is acquired under equal circumstances as the transmission spectrum of step M4), with the exception that no light is transmitted through the egg.

Preferred is also a method according to the present invention (or a method according to the present invention as described herein as being preferred, preferably a method of determining the sex of a chicken embryo inside the egg) wherein the method comprises step M6) and wherein determining the sex of the chicken embryo in step M6) is based on a spectral absorption function, wherein the spectral absorption function is determined based on the absorption spectrum (wherein the absorption spectrum is determined based on the transmission spectrum acquired in step M4) and a calibration spectrum, wherein the calibration spectrum is a measured spectrum of the light used for candling the egg in step M2), as defined above) by processing the absorption spectrum, preferably by taking a derivative of and/or by smoothing the absorption spectrum.

In the non-invasive method according to the present invention, excellent results in terms of determining the sex of an avian embryo inside the egg, preferably of a chicken embryo inside the egg, were achieved where eggs were used from a breed of avian species, preferably from a breed of chicken, that produces feathers of a certain colour for one sex and feathers of another (different) colour for the opposite sex (as defined herein or as defined herein as being preferred), and where a principal component analysis was performed for the spectral absorption function, as outlined in more detail below.

Preferred is thus a method according to the present invention (or a method according to the present invention as described herein as being preferred, preferably a method of determining the sex of a chicken embryo inside the egg) wherein the method comprises step M6) and wherein in step M6) the sex of the avian embryo, preferably of the chicken embryo, is determined by determining whether a combination value lies above or below a predetermined threshold, wherein the combination value refers to a combination of values of the spectral absorption function at different wavelengths,
wherein preferably the combination value refers to a linear combination of values of the spectral absorption function at different wavelengths, wherein the coefficients of the linear combination are determined from coefficients of one or more principal components resulting from a principal component analysis performed on spectral absorption functions determined for an ensemble of chicken embryos inside their eggs and wherein preferably the predetermined threshold is zero.

It has been found that analysing the spectral transmission and/or absorption spectrum measured for an egg in terms of its principal components as derived from a training dataset allows for a particularly efficient and yet reliable determination of properties of the egg. Using a principal component analysis in this way amounts to viewing a given measured spectrum, or some (discrete) function derived from it (such as the spectral absorption functions referred to herein), as a set, or list, of single-valued random variables {I_{λ}} corresponding to the measured (light) intensities (or the respectively derived values of the function derived from it) at the different wavelengths. The set or list of random variables may equivalently be viewed as a single multi-valued, i.e. vector-like, random variable. The underlying statistical ensemble is a given set of eggs whose properties are to be determined, wherein to each egg a certain spectrum, and hence corresponding values of the single-valued random variables (or, equivalently, a single “multi-value” of the multi-valued random variable), can be associated by measuring the respective spectrum. A property distinguishing some eggs from others will, as long as it has an effect on the optical properties of the egg, particularly its transmission characteristics, lead to variations in the spectra of the ensemble and therefore the values of the respective (single-valued) random variables corresponding to the spectrum. These variations can be captured in terms of the covariance matrix of the random variables {I_{λ}}. While a generic property will usually be reflected by variations in various parts of a spectrum, each of which might be relatively small and hence, per se, unsuitable as an indicator for the property to be determined, it is desirable to find an indicator which allows to reliably deduce the property to be determined from a given spectrum. Such an indicator will preferably be a quantity which varies as much as possible between spectra which differ in the respective property. Since a principal component analysis allows to sort linear combinations of the random variables {I_{λ}} according to their variance in the ensemble, a principal component analysis allows for an efficient choice of indicators to be derived from spectra in order to determine properties of the respective eggs based on them.

Preferably, the “different wavelengths” of the “combination of values of the spectral absorption function at different wavelengths”, as defined above (or below), are selected from the group of wavelength ranges consisting of:
the wavelength range from ≥720 nm to ≤760 nm
the wavelength range from ≥730 nm to ≤830 nm,
the wavelength range from ≥750 nm to ≤870 nm, preferably from >750 nm or 750 nm to ≤830 nm
   and
the wavelength range from ≥800 nm to ≤870 nm.

Preferred is furthermore a method according to the present invention (or a method according to the present invention as described herein as being preferred, preferably a method of determining the sex of a chicken embryo inside the egg), wherein the method comprises step M6) and wherein in step M6) the sex of the avian embryo, preferably of the chicken embryo, is determined based on a combination value, wherein the combination value refers to a combination of values of the spectral absorption function at different wavelengths,
wherein preferably the combination value is determined from one or more principal components of the spectral absorption function, wherein the one or more principal components refer to a principal component analysis performed on spectral absorption functions determined for an ensemble of avian embryos, preferably of chicken embryos, inside their eggs.

Also preferred is a method according to the present invention (or a method according to the present invention as described herein as being preferred, preferably a method of determining the sex of a chicken embryo inside the egg), wherein one or more principal components resulting from a principal component analysis are involved in determining the combination value and wherein at least one of the one or more principal components is selected from the group consisting of the first principal component and the third principal component,
wherein preferably
   the one or more principal components are the first principal component and the third principal component
and/or
   the one or more principal components do not comprise the second principal component.

The present invention also pertains to a system for non-invasively determining one or more properties of an avian egg (preferably of a chicken egg) and/or one or more properties of an avian embryo inside the egg (preferably of a chicken embryo inside the egg), more preferably for non-invasively determining the sex of a chicken embryo inside the egg, the system comprising at least the following elements:
S1) a light source for candling the egg with light having a spectrum extending at least over the range of wavelengths from ≥700 nm to ≤900 nm,
S2) light capturing means (preferably one or more than one light capturing means) for capturing transmitted light, wherein the captured transmitted light is a portion of the light for candling the egg having a spectrum as defined in element S1), wherein the portion is transmitted through the egg,
S3) a spectrometer for acquiring a transmission spectrum of the captured transmitted light as defined in element S2), wherein the transmission spectrum is based on one or more distinctive wavelength ranges and the one or more distinctive wavelength ranges are in each case predefined wavelength subranges of the range of wavelengths from ≥700 nm to ≤900 nm
and
S4) a determination unit
   for determining the one or more properties of the avian egg, preferably of the chicken egg, and/or the one or more properties of the avian embryo, inside the egg, preferably of the chicken embryo inside the egg,
      more preferably for determining the sex of the chicken embryo inside the egg,
   based on the transmission spectrum.

Generally, all aspects of the present invention discussed herein in the context of the non-invasive method of determining one or more properties of an avian egg and/or one or more properties of an avian embryo inside the egg apply mutatis mutandis to the system for non-invasively determining one or more properties of an avian egg and/or one or more properties of an avian embryo inside the egg, as defined here above.

Preferably, the determination unit of the system according to the present invention (element S4)) is or comprises a data processing unit which is connected with the spectrometer (element S3)) and preferably with the light source (element (S1)). The data processing unit is preferably adapted for performing at least the steps M5), comparing the transmission spectrum of the transmitted light acquired in step M4), and M6), determining the one or more properties of the avian egg and/or the one or more properties of the avian embryo inside the egg based on the transmission spectrum acquired in step M4), of the non-invasive method of the present invention, including all variants and preferred variants of steps M5) and M6) of the non-invasive method of the present invention, as disclosed herein. Preferably, the data processing unit comprises a software suitable for the purposes according to the present invention.

Preferred is a system according to the present invention (or a system according to the present invention as described herein as being preferred), wherein
the light capturing means S2) is adapted to capture the transmitted light (preferably only) within a defined measuring spot on the egg's surface, wherein preferably the measuring spot
   has a diameter in the range from ≥0.5 to ≤2.5 cm, preferably from ≥1 to ≤2.3 cm;
and/or
   extends over an area in the range from ≥0.2 to ≤5 cm², preferably from ≥0.8 to ≤4 cm² on the egg's surface.
      and/or
the spectrometer S3) comprises a detector, wherein the spectrometer's detector
   is adapted to detect the transmitted light irrespective of its origin;
and/or
   is adapted to detect the transmitted light without spatially resolving the transmitted light;
and/or
   is selected from the group consisting of a charge coupled device detector and a photo diode array detector.

Where the spectrometer (S3)) of the system according to the present invention comprises a detector which is adapted to detect the transmitted light irrespective of its origin, said transmitted light is preferably a portion of the light for candling the egg and having a spectrum as defined for the light source (cf. element S1)), wherein the portion is transmitted through the egg (as defined above for element S2)).

Preferably, the light capturing means of the system according to the present invention (cf. element S2)) comprises a lens, preferably an optical lens, for focusing or directing the captured light to the light guiding means (as described below) or to the spectrometer.

Also preferred is a system according to the present invention (or a system according to the present invention as described herein as being preferred), comprising as further element or as further elements
S5) light guiding means (preferably one or more light guiding means) for guiding the captured transmitted light from the light capturing means S2) to the spectrometer S3),
   wherein preferably the light guiding means comprises (or comprise) one or more optical fibres adapted or optimized for transmitting light of a wavelength in the range from ≥700 nm to ≤900 nm, preferably from ≥720 nm to ≤870 nm, more preferably from ≥750 nm to ≤870 nm and even more preferably from >750 nm or 750 nm to ≤870 nm;
and/or
S6) a carrier, preferably a carrier rack, having a plurality of compartments, wherein each compartment is configured for receiving an avian egg, preferably a chicken egg, and the compartments are separated from each other by partition walls for reducing the amount of scattered light,
   wherein preferably the carrier (or carrier-rack) is configured for allowing candling an egg (preferably a chicken egg) placed in a compartment and for allowing capturing light transmitted through the egg with the light capturing means,
      wherein more preferably the carrier (or carrier-rack) is configured for allowing light-tight coupling of the light source or of the light guiding means to an egg (preferably to a chicken egg).

Preferred is furthermore a system according to the present invention (or a system according to the present invention as described herein as being preferred), wherein the light source (element S1)) comprises or is a halogen lamp, preferably a tungsten halogen lamp, wherein the halogen lamp has
a power of ≥35 W, preferably of ≥40 W, more preferably of ≥50 W, and preferably ≤75 W;
   and/or
a luminous intensity of ≥1000 cd, preferably of ≥1100 cd, more preferably of ≥1200 cd and even more preferably of ≥1300 cd.

Also preferred is a system according to the present invention (or a system according to the present invention as described herein as being preferred), wherein the spectrometer S3) is selected from the group consisting of a Multi Channel Spectrometer, a Compact Grating Spectrometer and a Monolithic Miniature Spectrometer. More generally, it might be preferred to use as spectrometer S3) a spectrometer resolving the incoming light only spectrally, but not spatially. In other words, the spectrometer S3) does not necessarily need to be suitable for providing an image, particularly a spectral image, based on the incoming light. The light capturing means, the light guiding means and/or the spectrometer are preferably adapted to the distinctive wavelength ranges referred to herein.

For a skilled person, it was particularly surprising that a very high accuracy of predicting the sex of a chicken embryo inside the egg (where the chicken egg was from a breed of chicken that produces feather colour differentiation of the chicken, based on the sex of the chicken) could be achieved with such relatively simple spectrometers as used in the present invention (i.e. a Multi-Channel Spectrometer, a Monolithic Miniature Spectrometer or a Compact Grating Spectrometer). Similar methods known from the prior art had usually taught that a relatively complex hyperspectral camera was necessary for the respective purpose.

For an industrial application aiming at high throughput, a spectrometer with sufficient light sensitivity is beneficial. It has been found in own experiments that a Multi Channel Spectrometer (MCS) or a Compact Grating Spectrometer (CGS) excellently fulfil the respective requirements. For furthering industrial applicability, the present authors have found it beneficial to use a spectrometer fitted with a charge-coupled device sensor and/or a spectrometer or spectrometer system with ≥2 channels, preferably with ≥5 channels, and more preferably with ≥5 channels and ≤12 channels, even more preferably with 10 channels.

Preferred is therefore a system according to the present invention (or a system according to the present invention as described herein as being preferred), wherein
the spectrometer (element S3)) is or comprises
   a spectrometer selected from the group consisting of a Multi Channel Spectrometer and a Compact Grating Spectrometer,
or
   a spectrometer system comprising one or more spectrometers selected from the group consisting of a Multi Channel Spectrometer and a Compact Grating Spectrometer,
      wherein preferably
the spectrometer and/or the spectrometer system comprises ≥2 channels, preferably ≥5 channels, more preferably ≥5 channels and ≤12 channels, and even more preferably 10 channels;
   and/or
the spectrometer or the one or at least one of the more spectrometers of the spectrometer system comprises one or more (preferably one) charge-coupled device sensors.

For example, in one preferred variant of the present invention, the system according to the present invention comprises as spectrometer (element S3)) a Multi Channel Spectrometer with 10 channels, which is fitted with one or more charge-coupled device sensors. For example, in a further preferred variant of the present invention, the system according to the present invention comprises as spectrometer (element S3)) a spectrometer system with 10 channels, comprising several Compact Grating Spectrometers, wherein all of the Compact Grating Spectrometers are fitted with charge-coupled device sensors.

Where the system according to the present invention comprises as spectrometer a spectrometer or a spectrometer system comprising two or more channels, preferably electronic multiplexing is applied for processing the two or more channels.

In one preferred variant of the system of the present invention, the system of the present invention (or a system according to the present invention as described herein as being preferred) is combined with, or comprises elements of, a device for examining hatching eggs as disclosed in document WO 2019/174661 A1. For example, the carrier of the system of the present invention may be designed in such variant like the carrier (3) as disclosed in document WO 2019/174661 A1 and the system according to the present invention may comprise an egg transporting unit which is designed like the egg transporting unit (12) as disclosed in document WO 2019/174661 A1. Such egg transporting unit may also be connected to the determination unit (element S4)) of the system of the present invention) to form a device or system for determining one or more properties of an avian egg (preferably a chicken egg) and/or one or more properties of an avian embryo (preferably of a chicken embryo) inside the egg, and for sorting the eggs according to the result of determining said one or more properties.

The present invention also pertains to the use of a spectrometer selected from the group consisting of a Multi Channel Spectrometer, a Compact Grating Spectrometer, a Monolithic Miniature Spectrometer and combinations thereof, in a system and/or in a method for non-invasively determining one or more properties of an avian egg, preferably a chicken egg, and/or one or more properties of an avian embryo, preferably a chicken embryo, inside the egg,
preferably for non-invasively determining the sex of an avian embryo, preferably a chicken embryo, inside the egg,
   wherein preferably the egg is a chicken egg, obtained from a breed of chicken that produces feather colour differentiation of the chicken, based on the sex of the chicken.

Generally, all aspects of the present invention discussed herein in the context of the non-invasive method of determining one or more properties of an avian egg and/or one or more properties of an avian embryo inside the egg and in the context of the system according to the present invention apply mutatis mutandis to the use of a spectrometer, as defined here above.

The present invention also pertains to a computer program for determining one or more properties of an avian egg and/or one or more properties of an avian embryo inside the egg based on a transmission spectrum acquired according to steps M1) to M4), particularly any of their respective specific realizations described herein, of the method as defined further above, wherein the computer program comprises instructions which cause a computer to carry out steps M5) and/or M6), particularly any of their specific realizations described herein, of the method as defined further above. The “computer” executing the computer program can also correspond to one or more data processing units. For instance, the computer program may be executed by the determination unit S4) of the system defined further above, which may then comprise the one or more data processing units.

A computer program may be stored/distributed on a suitable medium, such as an optical storage medium or a solid-state medium, possibly supplied together with or as part of other hardware, but may also be distributed in other forms, such as via the Internet or other wired or wireless telecommunication systems.

It is noted that the symbols ≥ and ≤ are used herein to specify lower and, respectively, upper limits of parameter ranges, particularly wavelength ranges, indicate that the respective limit is included in the specified range, whereas the symbols > and < indicate that the respective limit is not included.

### FIGURES

The invention is further explained and illustrated by the appended figures, as briefly explained here below:

FIG. 1 shows a part of a measurement equipment for carrying out the non-invasive method of determining one or more properties of an avian egg and/or one or more properties of an avian embryo inside the egg, according to the present invention. The following elements of a system for non-invasively determining one or more properties of an avian egg and/or one or more properties of an avian embryo inside the egg, according to the present invention, are shown: a carrier rack (element S6)) having a plurality of compartments, wherein each compartment is configured for receiving a chicken egg, and the compartments are separated from each other by partition walls for reducing the amount of scattered light. Furthermore, light capturing means (two detector heads above the carrier rack; element S2)) for guiding the captured transmitted light from the light capturing means to the spectrometer (element S3), not shown in FIG. 1) are shown.

FIG. 2 shows a detail of FIG. 1 wherein two chicken eggs from a brown layer line of hens (Lohmann Brown) are placed in two different compartments of the carrier rack (element S6)). The carrier rack is configured for allowing candling a chicken egg placed in a compartment (cf. openings at the bottom of the compartments for allowing mounting of light sources). Above the eggs in their compartments, two detector heads (light capturing means, elements S2)) are shown for capturing light which has been transmitted through the egg.

FIG. 3 shows a detail of the measurement equipment shown in FIG. 1. The carrier rack (element S2)) is not present in FIG. 3 to allow showing three halogen lamps (35 W) (light sources, elements S1)) located below the carrier rack, which are connected to a determination unit (element S4), not shown in FIG. 3). The halogen lamps can be shut by swivelling shutters. Above the halogen lamps, two detector heads are visible (light capturing means, elements S2)).

FIG. 4 shows two exemplary transmission spectra acquired with the measurement equipment shown in FIGS. 1 to 3. Transmission spectrum 401 was acquired for a chicken egg with a male embryo inside, and transmission spectrum 402 was acquired for a chicken egg with a female embryo inside.

FIG. 5 shows exemplary absorbance spectra computed from the transmission spectra shown in FIG. 4, wherein absorbance spectrum 501 was computed from transmission spectrum 401, and absorbance spectrum 502 was computed from transmission spectrum 402.

FIG. 6 shows exemplary absorbance spectra computed from transmission spectra acquired for a whole (training) ensemble of chicken eggs. Two spectral bands 601, 602 can be roughly identified, which are, however, not clearly separated. Spectral band 601 is made up of absorbance spectra computed for eggs containing male chicken embryos, and spectral band 602 is made up of absorbance spectra computed for eggs containing female chicken embryos.

FIG. 7 shows spectral absorption functions corresponding to processed versions of the absorbance spectra from FIG. 6, wherein the processing includes a smoothing and a forming of the 1st derivative. Spectral band 701 is formed by processing the absorbance spectra in band 601, and spectral band 702 is formed by processing the absorbance spectra in band 602. By comparing FIG. 7 with FIG. 6, it can be seen that the separability of the spectral bands has increased by virtue of the processing, at least over the major part of shown wavelength range.

FIG. 8 shows exemplarily the loadings of the first principal component (“PC-1”) determined from the spectral absorption functions, i.e. the processed absorbance spectra, shown in FIG. 7.

FIG. 9 shows exemplarily the loadings of the third principal component (“PC-3”) determined from the spectral absorption functions, i.e. the processed absorbance spectra, shown in FIG. 7.

FIG. 10 shows a point cloud in which each point corresponds to a spectral absorption function, i.e. processed absorbance spectrum, represented in terms of its first and third principal component. The round (“f”) dots and the triangle shaped (“m”) dots belong to the absorbance spectral bands 702 and 701, respectively, shown in FIG. 7. While these absorbance spectra lay the basis for training, i.e. for determining the loadings of the principal components by performing a principal component analysis, the square-shaped dots (“0”) correspond to spectral absorption functions, i.e. processed absorbance spectra, determined analogously as for the absorbance spectra in bands 701, 702 used for training, but wherein the underlying transmission spectra have been acquired with eggs for which the sex of the chicken embryos contained therein was not known, thereby forming a control, or test, group. The line 1001 shown in FIG. 10 is chosen such that it best separates the round (“f”) dots from the triangle shaped (“m”) dots, and therefore preferably also splits the square-shaped (“0”) dots well into “female dots” and “male dots”.

FIG. 11 shows exemplary transmission spectra 1101, 1102, 1103 acquired for chicken eggs that have been incubated for different periods of time, indicating a significant decrease in the overall light transmission through the eggs as the embryos develop inside the eggs.

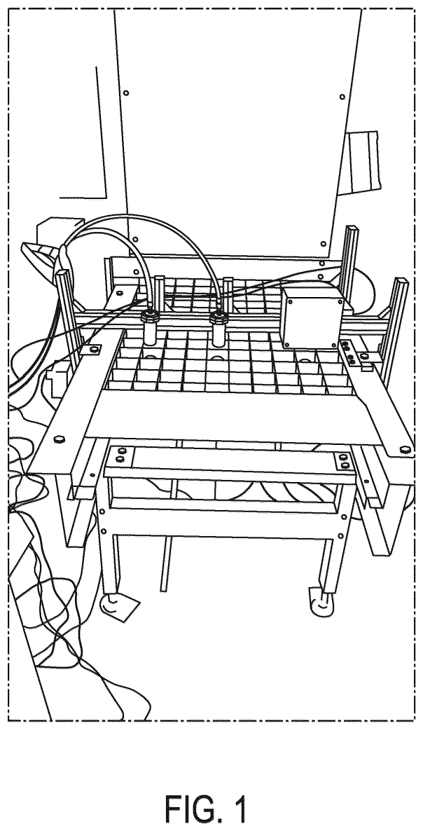

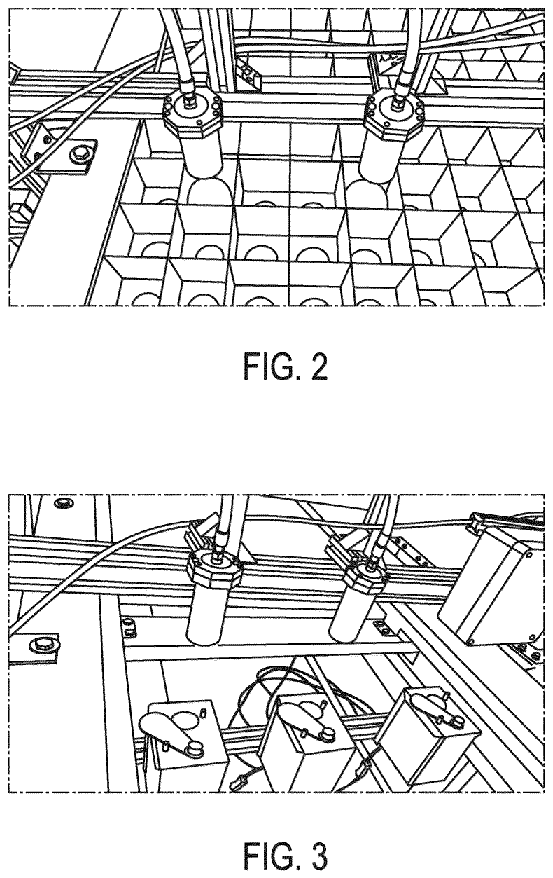

### EXAMPLES

The following examples shall further explain and illustrate the present invention without limiting its scope.

Brown chicken eggs were obtained from Lohmann Tierzucht GmbH and used for all experiments in the present examples. All eggs were from breeds of chicken (brown layer lines) that produced brown or brownish down feathers for female day-old chicks and white or yellowish down feathers for male day-old chicks. The sex of the chicks could thus (also) be determined or confirmed based on their feather colour after hatching.

The following measuring systems were used for the experiments of the examples in this section:

### Measuring System 1:

Spectrometer: Multi Channel Spectrometer (“MCS”; e.g. MCS FLEX CCD by Carl Zeiss) with CCD (“charge coupled device”) sensor;
   Wavelength range: 190-980 nm;
   2 channels, integration times: channel 1: 500 ms, channel 2: 1500 ms
Light guiding means: Near IR Mono 600 μm fibre optic cable.

### Measuring System 2:

Spectrometer: Monolithic Miniature Spectrometer (MMS; e.g. by Carl Zeiss) with PDA (“photo diode array”) sensor;
   Wavelength range: 300-1100 nm;
   2 channels, integration time: 10000 ms, 1×;
Light guiding means: Near IR Mono 600 μm fibre optic cable.

A 35 W standard halogen lamp with cold-light reflector (Osram) was used as light source in all experiments.

### Example 1: Optimization of Experimental Parameters (Including “Training Runs”)

### A. Experimental Set-Up:

The measurement equipment used in the experiments in the present examples comprised a carrier-rack having a plurality of compartments for placing eggs where the compartments were separated from each other by partition walls. Eggs were placed in the compartments and candled from below, using a light source (as specified above). Above the eggs, a detector head was placed for capturing the light transmitted through the egg. The diameter of the measuring spot on the egg's surface allowed by the detector head was about 2 cm. It was found that this relatively large diameter of the measuring spot reduced the sensitivity of the measurement against (occasionally occurring) inaccurate positioning of the eggs in the carrier-rack compartments. The detector head was connected via light guiding means (as specified above) to a spectrometer (Measuring System 1, as specified above) for acquiring a transmission spectrum of the captured transmitted light. The spectrometer and the light source were connected to a data processing unit.

Before the actual measurements were conducted, calibration spectra were taken and dark current measurements were performed for improving the actual measuring quality.

Calibration spectra were taken as a basis for the absorbance spectra of the eggs to be measured. To this end, the spectrum of the light source without a sample (egg) was measured and compared to the spectra of the sample (egg) to determine the absorbance of the sample. For measurement of the calibration spectra, light intensity was reduced to intensities processible for the spectrometers by use of neutral glass filters (Schott NG4 and Schott NG9)

### B. Measuring of the Spectra:

1191 chicken eggs that had been incubated for a period of 13 to 14 days were taken from the incubator, marked for later identification, and placed in the compartments of the carrier-rack (40 eggs per carrier-rack). While the eggs were candled from below with the light source, the light transmitted through the egg was captured within a defined measuring spot on the egg's surface (diameter of the measuring spot about 2 cm), transmitted to the spectrometer (Measuring System 1) via the light-guiding means (fibre optic cable) and recorded. The transmission spectra so received were further analysed as explained below.

C. Confirmation of the Sex of the Chicken after Hatching

After the measurements had been conducted (as explained under item B. above), the eggs were further incubated until the chicks hatched and the sex of the chicken was determined after hatching by methods known in the art for control. The following results were found:
Group 1—male chicks: 428
Group 2—female chicks: 447
Group 3—no egg: 5
Group 4—not hatched: 103
Group 5—control group (sex of chicks only revealed after measuring): 208

The information so obtained was then used for further analysing the spectra received.

### D. Analysis of the Spectra (as Taken According to the Method Described Under Item B. Above):

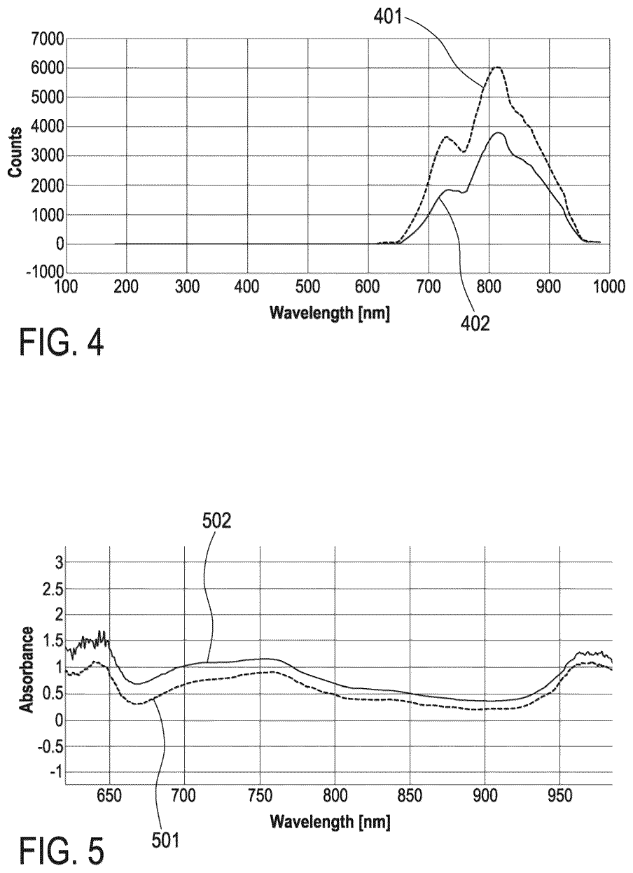

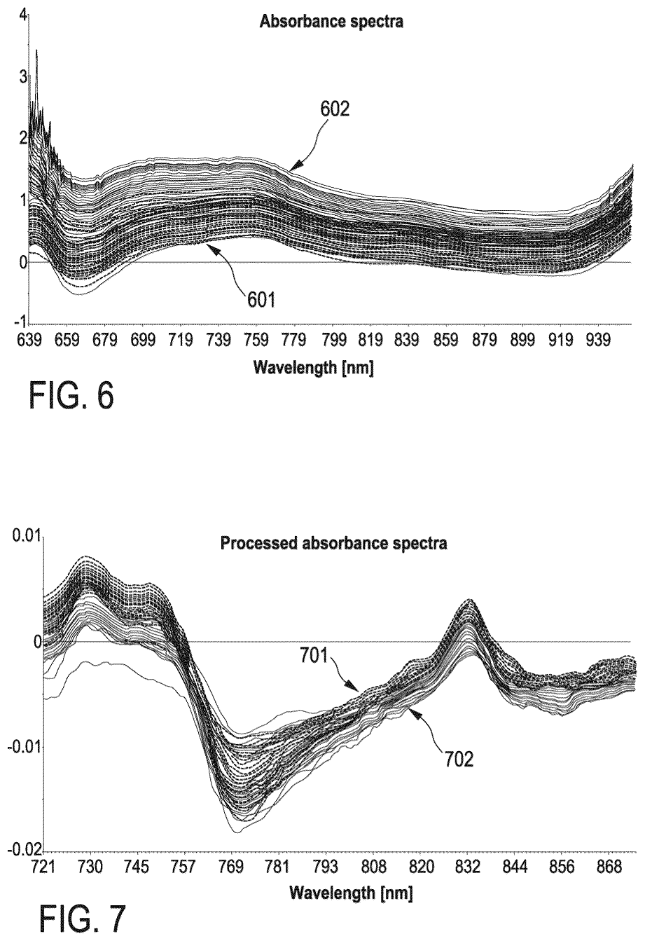

D.1 The measured transmission spectra were obtained as separate files in ASCII format. For analysis, they were imported into a data analysis software (“Unscrambler” by Camo Analytics). Then, a reference data set with respective known transmission spectra of male embryos inside their eggs and with known transmission spectra of female embryos inside their eggs was also added. In the raw spectra so received, the transmission in the spectral range between 620 nm and about 980 nm was considered but the spectral range between 180 nm and 620 nm was not further considered. It was found that raw spectra obtained from eggs containing male embryos showed a tendency for higher light transmission than spectra obtained from eggs containing female embryos.
D.2 A training data set was then created from combined data of Groups 1 and 2 (see above) and the respective absorbance spectra were computed (from the raw transmission spectra and the calibration spectra, see above). For the absorbance spectra, a tendency was observed fora higher light absorption of eggs containing female embryos compared with eggs containing male embryos, without a clear separation between the two groups. Two exemplary raw transmission spectra are shown in FIG. 4, wherein spectrum 401 refers to an egg containing a male chicken embryo and spectrum 402 refers to an egg containing a female chicken embryo. FIG. 5 shows the absorbance spectra computed from the transmission spectra shown in FIG. 4 in substantially the confined wavelength region where transmission is observed. Absorbance spectrum 501 is computed from transmission spectrum 401, i.e. corresponds to the egg containing the male chicken embryo, and absorbance spectrum 502 is computed from transmission spectrum 402, i.e. corresponds to the egg containing the female chicken embryo.
D.2.1 The absorbance spectra were then smoothed and the 1st derivation thereof was formed according to Savitzky-Golay. More precisely, in this exemplary analysis, the absorbance spectra were first smoothed according to Savitzky-Golay, wherein then the 1st derivation of the respective smoothed spectra was formed. This resulted in a much improved separation of data points received for eggs containing female embryos vs. for eggs containing male embryos, as can be seen by comparing FIG. 6, which shows the absorbance spectra corresponding to the training data set, and FIG. 7, which shows the 1st derivatives of the smoothed absorbance spectra, i.e. the processed absorbance spectra, which might generally be referred to as spectral absorption functions. While in FIG. 6 the spectral band 601 of spectra corresponding to the eggs containing male chicken embryos substantially overlaps with the spectral band 602 of spectra corresponding to the eggs containing female chicken embryos, in FIG. 7 the spectral band 701 of processed spectra corresponding to the eggs containing male chicken embryos only substantially overlaps with the spectral band 702 of processed spectra corresponding to the eggs containing female chicken embryos in a relatively narrow, intermediate range of wavelengths around approximately 775 nm. Similar results were received in respective experiments using Measuring System 2 (as defined above).
D.2.2 For further analysis, spectral ranges were defined according to features of the spectra (position of spectral bands) and tested for the quality of sex determination of the chicken embryos in the eggs based thereon. In a first analysis, the spectral ranges in the range from ≥620 nm to ≤980 nm, from ≥810 to ≤850 nm, from ≥800 to ≤870 nm and from ≥820 nm to ≤840 nm were included. Subsequently, a principal component analysis was performed for the spectral ranges from ≥620 nm to ≤980 nm, from ≥810 to ≤850 nm, from ≥800 to ≤870 nm and from ≥820 nm to ≤840 nm. In first approximation, the best distinction between the group of eggs containing female embryos vs. the group of eggs containing male embryos was found in the spectral range from ≥800 nm to ≤870 nm, as can already be appreciated from FIG. 7, where the separation between the spectral bands 701, 702 is particularly clear in this range. The distinction between sexes lay in this case on the first principal component, which indicates that the largest variances in optical properties among the eggs in Groups 1 and 2, on which the training data set was based, were due to the sex of the chicken embryos contained in the eggs.

While the first principal component may, thus, in this case be regarded as a suitable indicator for the sex of the chicken embryo inside the respective egg, further principal components may have a relevance for determining the sex of the chicken embryo inside the respective egg, too, or for determining other properties associated with the respective egg. For instance, it has been found that the loadings of the seventh principal component, i.e. the coefficients of the linear combination forming the seventh principal component from the respective spectral intensities, are still non-negligible and particularly peak, for instance, around 815 nm. Hence, the seventh principal component could still be used as an indicator for any property affecting the transmission and/or absorption characteristics of an egg at a wavelength around 815 nm. In fact, the acquired transmission spectra for Groups 1 and 2 have their main peaks near this wavelength, as can be seen exemplarily in FIG. 4, so that also a combination of the first and the seventh principal component was considered for determining the sex of chicken embryos inside their eggs.

Using a principal components indicative of the property of an egg or a chicken embryo inside the egg to be determined, the corresponding (single) value can be used as representative for the respective spectrum for the purpose of determining the property. Thus, once the loadings of the principal component(s) indicative of the property of interest have been determined from a training data set, an analysis of whole spectra can be avoided in favour of an analysis of only one or a few associated (single-valued) principal components, which allows for an increased computational efficiency.

For determining the sex of chicken embryos inside their eggs, for instance, only the first principal component of the respective spectrum could be considered, wherein the sex could be determined depending on whether this component lies above or below a predetermined threshold. For a generic ensemble of eggs, it might be assumed that the sex of the chicken embryos inside the eggs is approximately equally distributed, such that the predetermined threshold may be zero. More generally, for instance, for determining the sex of chicken embryos inside their eggs, the first and the seventh principal component of the respective spectrum could be considered, wherein the sex could be determined depending on whether a combination, such as a linear combination of the first and the seventh principal component, lies above or below a straight line in the plane spanned by the first and the seventh principal component, the line being positioned based on training data. The latter may in many cases be seen as being equivalent to determining whether the ratio of the first and the seventh principal component, one of the two possibly being shifted suitably beforehand, lies above or below a predetermined threshold.

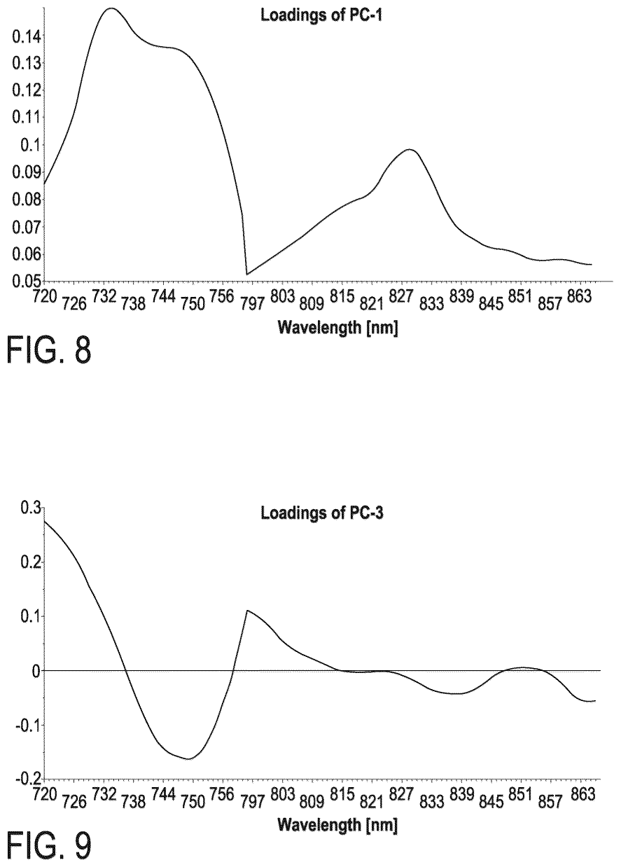

During the analysis it has been found that the second principal component might not be a good indicator for the sex of the chicken embryos, wherein instead it was expected that the second principal component was indicative of variations in the transmission spectra due to the different measuring channels used, each channel being associated in this case with a different spectral sensor. In fact, it might generally be preferred to determine the one or more properties to be determined not based on wavelength subranges or principal components which are known to be associated with variations in the acquired transmission spectra not indicative of the property to be determined, but possibly indicative of known characteristics of the measurement procedure applied.
D.2.3 With the results from the spectra analysis according to the method under this item D.2, a preliminary estimation of the probable prediction accuracy for the sex of a chicken embryo inside the egg was made for Groups 1 and 2 based on the information from the first principal component. According to this preliminary estimation, the following accuracy for a correct classification was predicted:
   Male embryos: 420 of 428 classified correctly as male, 8 incorrectly classified as female, corresponding to about 98% classified correctly as male.
   Female embryos: 422 of 447 classified correctly as female, 25 incorrectly classified as male, corresponding to about 94% classified correctly as female.
D.3 For a further optimized spectra analysis, a further training data set was created as explained under item D.2 above for the eggs from groups 1 and 2, and the respective absorbance spectra were computed, smoothed and the 1^{st} derivation thereof was formed according to Savitzky-Golay.
D.3.1 Spectral ranges were then defined as explained under item D.2.2 above and tested for the quality of sex determination based thereon, where the spectral ranges included in this experiment were (i) the range from ≥720 nm to ≤760 nm and (ii) the range from ≥800 nm to ≤870 nm. A significantly improved separation of data points received for eggs containing female embryos vs. for eggs containing male embryos was thus found. The spectral range from ≥730 nm to ≤830 nm and the spectral range from ≥750 nm to ≤870 nm, preferably from >750 nm or 750 nm to ≤830 nm, and specifically the spectral region (i.e., for instance, of about ±15 nm, ±10 nm or ±5 nm) around a wavelength of 750 nm comprised particularly significant information on sexes of the chicken embryos inside their eggs. This information corresponds to the general absorption around the characteristic peaks in the transmission spectra, as exemplified in FIG. 4, and specifically the absorption responsible for the dip in between the peaks.
D.3.2 A principal component analysis was performed for the spectral ranges of the further optimized spectra analysis according to items D.3.1 and D.3.2. The resulting loadings found for the first and the third principal component are shown in FIGS. 8 and 9, respectively. The magnitude of the respective loadings indicate that the first principal component can be substantially associated with a sex-specific general absorption between ≥730 nm to ≤830 nm, as particularly manifested around the two characteristic peaks in this region of the transmission spectra exemplarily seen in FIG. 4, and that the third principal component can be substantially associated with the dip in between the two characteristic peaks at about a wavelength of 750 nm. In this optimized analysis it has been found that the sex-specific information can be particularly well displayed in terms of a combination of principal components 1 and 3, thus meaning a further improvement over the results from the experiment explained under item D.2 above. While principal component 1 has been found in this analysis to represent the generally higher absorption of eggs containing female embryos compared to eggs containing male embryos, principal component 3 has been found in this analysis to represent a further absorption characteristic distinguishing eggs containing female embryos from eggs containing male embryos.

When combining principal components to arrive at an indicator for the property to be determined, the property may be determined based on whether the combination of principal components lies above or below a predetermined threshold. Generally, principal components can be combined in any way to form a suitable indicator. For instance, the property can be determined based on a linear combination of principal components.

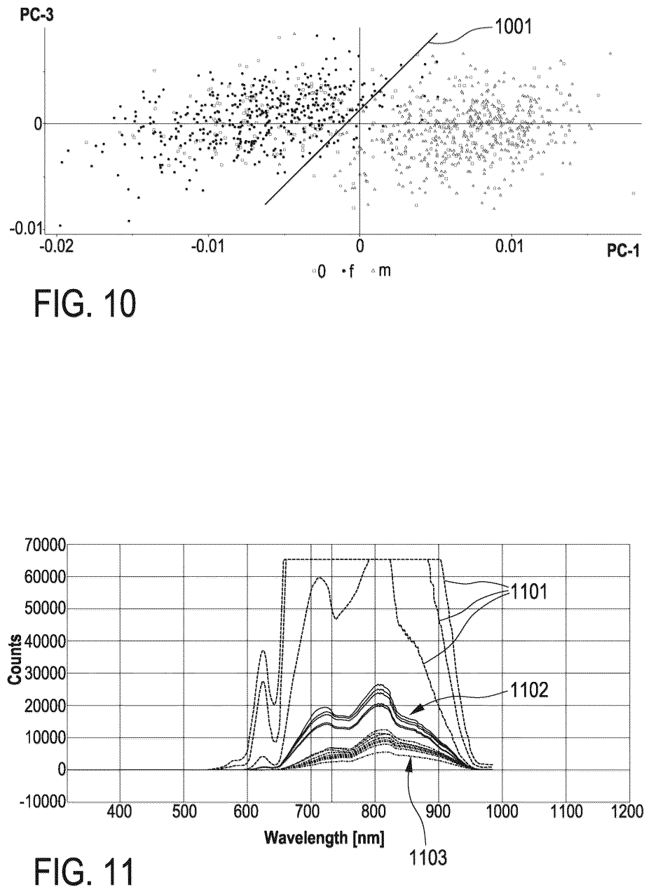

As a consequence of the optimized analysis, it has been found that it might be preferred that, for determining the sex of chicken embryos inside their eggs, the first and the third principal component of the respective spectrum could be considered, wherein the sex could be determined depending on whether a combination, such as a linear combination of the first and the seventh principal component, lies above or below a straight line in the plane spanned by the first and the third principal component, the line being positioned based on training data. The latter may in many cases be seen as being equivalent to determining whether the ratio of the first and the third principal component, one of the two possibly being shifted suitably beforehand, lies above or below a predetermined threshold.
D.3.3 With the results from the spectra analysis according to the method under this item D.3, an optimized estimation of the probable prediction accuracy for the sex of a chicken embryo inside the egg was made for Groups 1 and 2, including the information from the first and third principal component (i.e. principal component 1 and 3, as stated above), as further detailed below with reference to FIG. 10. According to this optimized estimation, the following accuracy for a correct classification was predicted:
   Male embryos: 415 of 428 classified correctly as male, 13 incorrectly classified as female, corresponding to about 97% classified correctly as male.
   Female embryos: 425 of 447 classified correctly as female, 22 incorrectly classified as male, corresponding to about 95% classified correctly as female.
   Overall: 840 of 875 classified correctly as male or female, corresponding to about 96% correctly classified eggs.

### Example 2: Prediction of the Sex of Chicken Embryos Inside the Egg of a Control Group According to the Method of the Invention (“Validation Run”)

The sex of the chicken embryos inside the eggs of Group 5 (control group) was predicted according to the method described under item D.3 above, as further detailed below with reference to FIG. 10. The result of the prediction was then compared to the sex of the chicks as confirmed after hatching. The following accuracy of classification of the sex of the chicken was found according to the prediction:
Male embryos: 96 of 103 classified correctly as male, 7 incorrectly classified as female, corresponding to about 93% classified correctly as male.
Female embryos: 101 of 105 classified correctly as female, 4 incorrectly classified as male, corresponding to about 96% classified correctly as female.
Overall: 197 of 208 classified correctly as male or female, corresponding to about 95% correctly classified eggs.

FIG. 10 shows the first and the second principal component, as resulting from the principal component analysis performed on the training data set for each of the corresponding spectral absorption functions, i.e. processed absorbance spectra, plotted against each other. While triangle-shaped dots correspond to spectra computed for the training data from Group 1 (i.e., belonging to eggs with male embryos inside) and round dots correspond to spectra computed for the training data from Group 2 (i.e., belonging to eggs with female embryos inside), the plot in FIG. 10 also includes square-shaped dots, which correspond to the spectral absorption functions, i.e. processed absorbance spectra, computed for the data measured for Group 5 (i.e., belonging to eggs for which it is not known whether they have a male or a female embryo inside). The principal components for these latter spectra, which might be regarded as test or control spectra, are computed with the loadings learned from the principal component analysis performed for the training data. The dividing line 1001 shown in FIG. 10 represents the straight line best separating the triangle-shaped dots from the round dots, wherein the best separation may generally refer to a sum of absolute distances or squared distances of the respective dots to the straight line. The line being tilted indicates that both the first and the third principal component are needed for an optimized separation between triangle-shaped and round dots and therefore an optimized distinction between eggs containing male embryos and eggs containing female embryos. The offset of the line from the origin mainly reflects the circumstance that Groups 1 and 2 of the eggs are not equal in size. The sex of the embryos inside the eggs of Group 5 (control group), represented by the square-shaped dots in FIG. 10, can be predicted based on the side of the line on which the respective square-shaped dot lies. Determining the side of the line on which a point lies might generally also be viewed, namely in a rotated coordinate system, as determining whether the point lies above or below a predefined threshold given by the line. In this particular case shown in FIG. 10, it could be predicted that square-shaped dots lying to the lower right side of the line correspond to eggs with male embryos inside, while square-shaped dots lying to the upper left side of the line correspond to eggs with female embryos inside. Hence, the offset from the origin and the slope of the line 1001 indicate the linear combination of the first and the third principal component that is chosen as an indicator for the sex of the embryos inside the eggs.

From the results of Examples 1 and 2 it can be seen that excellent results in terms of prediction of the sex of chicken embryos inside their eggs can be received, if the eggs are obtained from a breed of chicken that produces brown or brownish feathers for one sex and white or yellowish feathers for the opposite sex, and where a Multi-Channel Spectrometer (preferably with CCD sensor) or a Monolithic Miniature Spectrometer (preferably with PDA sensor) is used for acquiring a transmission spectrum in step M3). Similar results can be expected according to preliminary experiments performed by the authors where a Compact Grating Spectrometer (preferably with CCD sensor) is used for acquiring a transmission spectrum in step M3).

This result is surprising since a skilled person would not have assumed that an accuracy of predicting the sex of a chicken embryo inside the egg as high as was found in the present experiments could be achieved with the relatively simple spectrometers used in the present experiments (i.e. a Multi-Channel Spectrometer, a Monolithic Miniature Spectrometer or a Compact Grating Spectrometer). In similar experiments which had been performed earlier, a relatively complex hyperspectral camera was used for the respective purpose.

From the results of Examples 1 and 2 it can also be seen that the optimized method according to item D.3 of Example 1 allows a more accurate prediction of the sex of a chicken embryo inside the egg than e.g. the preliminary method according to item D.2 of Example 1. In particular, it might therefore be preferred that a combination of the first and the third principal component of the respectively obtained spectra is used for predicting the sex of a chicken embryo inside the egg, wherein the loadings of the principal components may preferably be determined from a principal component analysis of corresponding spectra obtained for training data.

### Example 3: Identification of Immature Chicken Embryos Inside the Egg According to the Method of the Invention

Chicken eggs with immature chicken embryos of different types were obtained:

A first group of eggs was incubated for less than one day so that the chicken embryos inside the eggs were scarcely developed at the time the measuring according to the non-invasive method of the present invention was conducted.

A second group of eggs contained chicken embryos whose development status was equivalent to about 9 to 10 days of incubation at the time the measuring according to the non-invasive method of the present invention was conducted.

Transmission spectra were then recorded of the eggs of both groups as explained in items A. and B. of Example 1 above.

For the first group of eggs (see above) it was found that, due to an extremely high light transmission, the spectrometer overdrove. This was noticeable by a horizontal line in the spectrum. A very similar result was found when unfertilized eggs where used in the experiment instead of eggs which were incubated for less than one day.

For the second group of eggs it was found that the light transmission observed for an egg containing a chicken embryo whose development status was equivalent to about 9 to 10 days of incubation was much higher than the light transmission observed for an egg containing a chicken embryo whose development status was equivalent to about 13 to 14 days of incubation. On the other hand, the light transmission observed for an egg containing a chicken embryo whose development status was equivalent to about 9 to 10 days of incubation was much lower than for an egg that had been incubated for less than one day, or for an unfertilized egg.

Hence, the first group of eggs, the second group of eggs and eggs containing chicken embryos whose development status was equivalent to about 13 to 14 days of incubation could be distinguished from each other by means of the light transmission they allow. The light transmission decreases as the embryo develops. The light transmission can be measured, for instance, in terms of predefined characteristics of the transmission spectra obtained for the respective eggs, such as a maximum, an average or an overall transmission, and/or whether the transmission spectrum comprises a horizontal line. For instance, the maximum transmission may correspond to a peak height in the respective transmission spectrum. A horizontal line in the transmission spectrum, which might be present in the spectrum instead of a peak, may indicate that the spectrometer overdrove due to the large transmission through the egg, indicating that the egg belongs to Group 1 (or that the egg is an unfertilized egg). FIG. 11 shows exemplary transmission spectra, wherein spectra 1101 correspond to eggs from Group 1 and/or unfertilized eggs, spectra 1102 correspond to eggs from Group 2 and spectra 1103 correspond to eggs containing a chicken embryo whose development status was equivalent to about 13 to 14 days.

It can therefore be seen from the results of the experiments of this Example 3 that eggs containing immature chicken embryos can easily and with high precision be identified by the method of the present invention. The method of the present invention is therefore excellently suited for detecting or determining an egg's fertilization state (fertilized or unfertilized), an (unhatched) chicken embryo's vitality inside the egg and an unhatched chicken embryo's state of development (inside the egg).

Moreover, the development state of an avian embryo, in particular of a chicken embryo, inside the egg can also be determined with the method according to the present invention. For example, the transmission spectrum of a chicken embryo inside the egg can be compared for this purpose to reference spectra of similar chicken embryos in different development states, e.g. in terms of different numbers of days of incubation after laying. From such comparison of spectra, the development state of the chicken embryo in the egg to be assessed can be determined with considerable precision.

We claim:

1. A non-invasive method of determining one or more properties of an avian egg and/or one or more properties of an avian embryo inside the egg, comprising at least the following steps:
M1) obtaining an avian egg,
M2) candling the egg obtained in step M1) with light having a spectrum extending at least over the range of wavelengths from ≥700 nm to ≤900 nm from a light source,
M3) capturing light transmitted through the egg, wherein the captured transmitted light is a portion of the light used for candling the egg in step M2),
M4) acquiring a transmission spectrum of the transmitted light captured in step M3) based on one or more distinctive wavelength ranges, wherein the one or more distinctive wavelength ranges are in each case predefined wavelength subranges of the range of wavelengths from ≥700 nm to ≤900 nm as defined in step M2),
and
M6) determining the one or more properties of the avian egg and/or the one or more properties of the avian embryo inside the egg based on the transmission spectrum acquired in step M4).

2. The method according to claim 1,
wherein the one or one of the more properties of the avian egg is selected from the group consisting of the egg's fertilization state and the germ load of the egg;
wherein the one or one of the more properties of the avian embryo inside the egg is selected from the group consisting of the avian embryo's state of development, the vitality of the avian embryo and the sex of the avian embryo;
and/or
comprising as additional step M5) the following step:
M5) comparing the transmission spectrum of the transmitted light acquired in step M4), or an absorption spectrum based on said transmission spectrum, in the one or more distinctive wavelength ranges with corresponding transmission spectra or with corresponding absorption spectra from a predefined database in the respective same one or more distinctive wavelength ranges, where the corresponding transmission spectra or the corresponding absorption spectra, particularly in the distinctive wavelength ranges, define known values of the one or more properties of the avian egg, preferably of the chicken egg and/or of the one or more properties of the avian embryo, preferably of the chicken embryo, inside the egg;
and/or
comprising as step M6) the following step:
M6) determining the one or more properties of the avian egg, preferably of the chicken egg, and/or the one or more properties of the avian embryo, preferably the chicken embryo, inside the egg, based on the transmission spectrum acquired in step M4) and/or based on the result of comparing the transmission spectrum of the transmitted light acquired in step M4), or the absorption spectrum based on said transmission spectrum, in the one or more defined distinctive wavelength ranges, with the corresponding transmission spectra or with the corresponding absorption spectra from a predefined database in the respective same one or more distinctive wavelength ranges, as defined in step M5).

3. The method according to claim 1,
wherein the method is a non-invasive method of determining the sex of a chicken embryo inside the egg,
preferably comprising
as step M1) the following step:
M1) obtaining an egg from a breed of chicken that produces feather colour differentiation of the chicken, based on the sex of the chicken,
and/or
as additional step M5) the following step:
M5) comparing the transmission spectrum of the transmitted light acquired in step M4), or an absorption spectrum based on said transmission spectrum, in the one or more distinctive wavelength ranges with corresponding transmission spectra or corresponding absorption spectra from a predefined database in the respective same one or more distinctive wavelength ranges, where the corresponding transmission spectra or the corresponding absorption spectra define known sexes of chicken embryos inside their eggs;
and/or
as step M6) the following step:
M6) determining the sex of the chicken embryo inside the egg based on the transmission spectrum acquired in step M4) and/or based on the result of comparing the transmission spectrum of the transmitted light acquired in step M4), or an absorption spectrum based on said transmission spectrum, in the one or more defined distinctive wavelength ranges, with corresponding transmission spectra or with corresponding absorption spectra from a predefined database in the respective same one or more distinctive wavelength ranges, as defined in step M5).

4. The method according to claim 1, wherein the egg obtained in step M1)
is obtained from a breed of chicken that produces brown or brownish feathers for one sex and white or yellowish feathers for the opposite sex;
and/or
is obtained from a breed of chicken which is a brown layer breed of chicken, preferably selected from the group consisting of a Hyline Brown breed of chicken, a Lohmann Brown breed of chicken and an ISA Brown breed of chicken;
and/or
is a hatching egg.

5. The method according to claim 1, wherein
the egg obtained in step M1) has been incubated for a period in the range from ≥9 to ≤15 days, preferably in the range from ≥12 to ≤14 days, more preferably in the range from ≥13 to ≤14 days after laying;
and/or
the light used for candling the egg in step M2) is light having a spectrum extending at least over the range of wavelengths from ≥720 nm to ≤870 nm, preferably from ≥750 nm or ≥750 nm to ≤870 nm.

6. The method according to claim 1, wherein the light transmitted through the egg is captured in step M3) within a defined measuring spot on the egg's surface, wherein the measuring spot
has a diameter in the range from ≥0.5 to ≤2.5 cm, preferably from ≥1 to ≤2.3 cm;
and/or
extends over an area in the range from ≥0.2 to ≤5 cm², preferably from ≥0.8 to ≤4 cm² on the egg's surface.

7. The method according to claim 1, wherein step M1) further comprises
providing a carrier, preferably a carrier rack, having a plurality of compartments, wherein each compartment is configured for receiving an avian egg, preferably a chicken egg, and the compartments are separated from each other by partition walls for reducing the amount of scattered light,
wherein preferably the carrier, preferably the carrier rack, is configured for allowing candling with a light source an avian egg, preferably a chicken egg, placed in a compartment; and for allowing capturing light transmitted through the egg;
and
placing the avian egg, preferably the chicken egg, in a compartment of the carrier, preferably of the carrier rack.

8. The method according to claim 1, wherein the light source for candling the egg in step M2) is a halogen lamp, preferably a tungsten halogen lamp, wherein the halogen lamp has
a power of ≥35 W, preferably of ≥40 W, more preferably of ≥50 W and preferably ≤75 W;
and/or
a luminous intensity of ≥1000 cd, preferably of ≥1100 cd, more preferably of ≥1200 cd and even more preferably of ≥1300 cd.

9. The method according to claim 1, wherein
the one or more distinctive wavelength ranges of step M4) are selected from the group consisting of
the wavelength range from ≥720 nm to ≤760 nm
the wavelength range from ≥730 nm to ≤830 nm,
the wavelength range from ≥750 nm to ≤870 nm, preferably from ≥750 nm or ≥750 nm to ≤830 nm
and
the wavelength range from ≥800 nm to ≤870 nm;
and/or
the method comprises as step M5) the following step:
M5) comparing the transmission spectrum of the transmitted light acquired in step M4) or an absorption spectrum based on said transmission spectrum in the one or more distinctive wavelength ranges, wherein the one or more distinctive wavelength ranges are selected from the group of wavelength ranges consisting of:
the wavelength range from ≥720 nm to ≤760 nm,
the wavelength range from ≥730 nm to ≤830 nm,
the wavelength range from ≥750 nm to ≤870 nm, preferably from ≥750 nm or ≥750 nm to ≤830 nm
and
the wavelength range from ≥800 nm to ≤870 nm,
with corresponding transmission spectra or corresponding absorption spectra from a predefined database in the respective same one or more distinctive wavelength ranges, where the corresponding transmission spectra or the corresponding absorption spectra define known sexes of chicken embryos inside their eggs;
and/or
the method comprises as step M6) the following step:
M6) determining the sex of the avian embryo, preferably of the chicken embryo, inside the egg, based on the result of comparing,
the transmission spectrum of the transmitted light acquired in step M4) in the one or more defined distinctive wavelength ranges or an absorption spectrum based on said transmission spectrum, in the one or more defined distinctive wavelength ranges, as defined in step M5),
with
corresponding transmission spectra or with corresponding absorption spectra defining known sexes of chicken embryos inside their eggs in the respective same one or more distinctive wavelength ranges, as defined in step M5).

10. The method according to claim 2, preferably according to claim 3, wherein the method comprises step M5) and wherein in step M6) determining the sex of the avian embryo, preferably the sex of the chicken embryo, is based on an absorption spectrum, wherein the absorption spectrum is determined based on the transmission spectrum acquired in step M4) and a calibration spectrum, wherein the calibration spectrum is a measured spectrum of the light used for candling the egg in step M2),
wherein preferably the transmission spectrum based on which the absorption spectrum is determined is corrected based on a dark current spectrum, wherein the dark current spectrum corresponds to a spectrum which is acquired under equal circumstances as the transmission spectrum of step M4), with the exception that no light is transmitted through the egg.

11. The method according to claim 10, wherein in step M6) the sex of the avian embryo, preferably of the chicken embryo, is determined by determining whether a combination value lies above or below a predetermined threshold, wherein the combination value refers to a combination of values of a spectral absorption function at different wavelengths, wherein the spectral absorption function is determined based on the absorption spectrum by taking a derivative of and/or by smoothing the absorption spectrum,
wherein preferably the combination value refers to a linear combination of values of the spectral absorption function at different wavelengths, wherein the coefficients of the linear combination are determined from coefficients of one or more principal components resulting from a principal component analysis performed on spectral absorption functions determined for an ensemble of chicken embryos inside their eggs and wherein preferably the predetermined threshold is zero,
and/or
wherein in step M6) the sex of the avian embryo, preferably of the chicken embryo, is determined based on a combination value, wherein the combination value refers to a combination of values of the spectral absorption function at different wavelengths,
wherein preferably the combination value is determined from one or more principal components of the spectral absorption function, wherein the one or more principal components refer to a principal component analysis performed on spectral absorption functions determined for an ensemble of avian embryos, preferably of chicken embryos, inside their eggs.

12. The method according to claim 11, wherein one or more principal components resulting from a principal component analysis are involved in determining the combination value and wherein at least one of the one or more principal components is selected from the group consisting of the first principal component and the third principal component,
wherein preferably
the one or more principal components are the first principal component and the third principal component
and/or
the one or more principal components do not comprise the second principal component.

13. A system for non-invasively determining one or more properties of an avian egg and/or one or more properties of an avian embryo inside the egg, preferably for non-invasively determining the sex of a chicken embryo inside the egg, the system comprising at least the following elements:
S1) a light source for candling the egg with light having a spectrum extending at least over the range of wavelengths from ≥700 nm to ≤900 nm,
S2) light capturing means for capturing transmitted light, wherein the captured transmitted light is a portion of the light for candling the egg having a spectrum as defined in element S1), wherein the portion is transmitted through the egg,
S3) a spectrometer for acquiring a transmission spectrum of the captured transmitted light as defined in element S2), wherein the transmission spectrum is based on one or more distinctive wavelength ranges and the one or more distinctive wavelength ranges are in each case predefined wavelength subranges of the range of wavelengths from ≥700 nm to ≤900 nm
and
S4) a determination unit
for determining the one or more properties of the avian egg, preferably of the chicken egg, and/or the one or more properties of the avian embryo, inside the egg, preferably of the chicken embryo inside the egg,
more preferably for determining the sex of the chicken embryo inside the egg,
based on the transmission spectrum,
wherein preferably
the spectrometer S3) is selected from the group consisting of a Multi Channel Spectrometer, a Compact Grating Spectrometer and a Monolithic Miniature Spectrometer.

14. Use of a spectrometer selected from the group consisting of a Multi Channel Spectrometer, a Compact Grating Spectrometer, a Monolithic Miniature Spectrometer and combinations thereof, in a system and/or in a method for non-invasively determining one or more properties of an avian egg, preferably a chicken egg, and/or one or more properties of an avian embryo, preferably a chicken embryo, inside the egg,
preferably for non-invasively determining the sex of an avian embryo, inside the egg,
wherein preferably the egg is a chicken egg, obtained from a breed of chicken that produces feather colour differentiation of the chicken, based on the sex of the chicken.

15. A computer program for determining one or more properties of an avian egg and/or one or more properties of an avian embryo inside the egg based on a transmission spectrum acquired according to steps M1) to M4) of the method as defined in claim 1, wherein the computer program comprises instructions which cause a computer to carry out steps M5) and/or M6) of the method if the program is executed by the computer.
